(19) United States
(12) Patent Application Publication
(10) Pub. No.: US 2021/0134414 A1
(43) Pub. Date: May 6, 2021

(54) GENERATION OF COMBINED INFORMATION DISPLAY INTERFACES TO SUPPORT CLINICAL PATIENT PRIORITIZATION

(71) Applicant: Seattle Children's Hospital d/b/a Seattle Children's Research Institute, Seattle, WA (US)

(72) Inventor: Ari H. Pollack, Seattle, WA (US)

(21) Appl. No.: 17/087,437

(22) Filed: Nov. 2, 2020

Related U.S. Application Data
(60) Provisional application No. 62/929,571, filed on Nov. 1, 2019.

(51) Int. Cl. G16H 15/00; G16H 50/70; G16H 10/60; G16H 40/20; G16H 10/40; G16H 50/50; G06F 3/14

(52) U.S. Cl. CPC G16H 15/00; G16H 50/70; G16H 10/60; G06F 3/14; G16H 10/40; G16H 50/50; G16H 40/20

(57) ABSTRACT

In some embodiments, techniques for generating a user interface for summarizing care trend information for multiple patients are provided. For each patient of a plurality of patients, treatment information is collected for the patient from an electronic health record system. The treatment information reflects at least one of a patient acuity, a patient clinical problem, and a clinical change in the patient. An overview presentation of the plurality of patients is generated. The overview presentation includes summarized information for each patient of the plurality of patients. The overview presentation is presented to a user. The overview presentation allows a user to prioritize care for the plurality of patients in a way that is superior to browsing individual patient records within the electronic health record system.

### CROSS-REFERENCE(S) TO RELATED APPLICATION(S)

This application claims the benefit of Provisional Application No. 62/929571, filed Nov. 1, 2019, the entire disclosure of which is hereby incorporated by reference herein for all purposes.

### BACKGROUND

Individual hospitalized patients have the potential to generate thousands of data elements each day. Clinicians need systems that organize and present this data efficiently to minimize the workload associated with finding, processing, and ultimately acting on clinical data. Although Electronic Health Records (EHRs) have been viewed as a solution to meet the needs of clinicians, they can introduce new sources of cognitive burden (CB). Clinicians must prioritize clinical care tasks, and ensure patients with the highest need are addressed first, but current EHRs offer little support for this critical prioritization process.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In some embodiments, a method of generating a user interface for summarizing care trend information for multiple patients is provided. For each patient of a plurality of patients, treatment information for the patient is collected from an electronic medical record system. The treatment information reflects at least one of a patient acuity, a patient clinical problem, and a clinical change in the patient. An overview presentation of the plurality of patients is generated. The overview presentation includes summarized information for each patient of the plurality of patients. The overview presentation is presented to a user.

In some embodiments, a non-transitory computer-readable medium is provided. The computer-readable medium has computer-executable instructions stored thereon that, in response to execution by one or more processors of a computing device, cause the computing device to perform actions for generating a user interface for summarizing care trend information for multiple patients, the actions comprising: for each patient of a plurality of patients, collecting treatment information for the patient from an electronic medical record system, wherein the treatment information reflects at least one of a patient acuity, a patient clinical problem, and a clinical change in the patient; generating an overview presentation of the plurality of patients, wherein the overview presentation includes summarized information for each patient of the plurality of patients; and displaying the overview presentation to a user.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram that illustrates a non-limiting example embodiment of a health care environment 100 according to various aspects of the present disclosure.

FIG. 2 is a flowchart that illustrates a non-limiting example embodiment of a method of summarizing care trend information for multiple patients according to various aspects of the present disclosure.

FIG. 3A illustrates a non-limiting example embodiment of an overview presentation organized by acuity according to various aspects of the present disclosure.

FIG. 3B illustrates a non-limiting example embodiment of detailed information presented after an interaction with a bar chart illustrated in FIG. 3A.

FIG. 4A illustrates a non-limiting example embodiment of an overview presentation organized by clinical problem, according to various aspects of the present disclosure.

FIG. 4B illustrates a non-limiting example embodiment of detailed information presented after an interaction with a segment associated with a specific clinical problem for a given patient in the clinical problem interface 402 illustrated in FIG. 4A.

FIG. 5A illustrates a non-limiting example embodiment of an overview presentation organized by clinical change, according to various aspects of the present disclosure.

FIG. 5B illustrates a non-limiting example embodiment of summarized information for a given patient presented after interaction with a clinical change circle for a given patient in the clinical change interface 502 illustrated in FIG. 5A.

FIG. 5C illustrates a non-limiting example embodiment of detailed information for a given patient presented after interaction with the summarized information for a given patient in the clinical change interface 502 illustrated in FIG. 5B.

FIG. 6 is a block diagram that illustrates a non-limiting example embodiment of a computing device appropriate for use as a computing device with embodiments of the present disclosure.

### DETAILED DESCRIPTION

In current electronic health record (EHR) systems, detailed information about patients is often segregated into records by patient. As such, multiple screens or pages are required to view and compare details from multiple patients in order to make care prioritization decisions. Further, while existing EHR systems may provide historical information within detailed test records for a given patient, these existing systems require multiple clicks or taps into a patient record in order to retrieve such information.

What is desired are improved interfaces that combine detailed information from multiple patients, including trend information, into a single-screen interface that can be easily navigated on a mobile device. The description below provides particular manners of collecting, summarizing, and presenting specific, limited information related to multiple patients, and the trends for that information for the patients over time. These techniques provide improved user interfaces for electronic devices, particularly those with small screens, at least because a large quantity of information can be displayed in a single screen without further navigation into detailed records, thus improving the speed of presenting, viewing, and comparing the information used for care prioritization. These improved user interfaces also eliminate the need for paging through multiple screens to collect similar information, which can be particularly useful on touch-interface devices such as tablets.

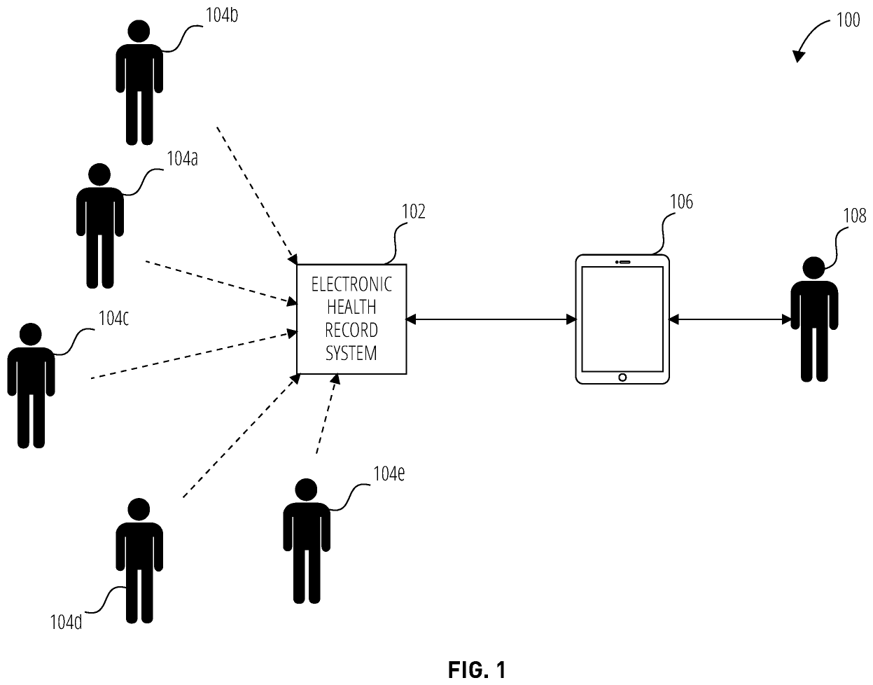

FIG. 1 is a schematic diagram that illustrates a non-limiting example embodiment of a health care environment 100 according to various aspects of the present disclosure. As shown, the health care environment 100 may be a hospital, an outpatient clinic, a telemedicine environment, a rehabilitation facility, or any other type of environment in which finite caregiving resources are prioritized.

The health care environment 100 includes a plurality of patients (including, but not limited to, patient 104a, patient 104b, patient 104c, patient 104d, and patient 104e). Caregivers within the health care environment 100 provide treatment, measure vital signs, diagnose conditions, and perform other healthcare-related tasks with relation to the patients. Information regarding the patients, including but not limited to the treatments, vital signs, and diagnosed conditions, is stored in an electronic health record system (EHR system 102). The EHR system 102 stores the information regarding the patients over time, such that the health histories of the patients (including but not limited to trends in vital signs, lab results, etc.) are stored and may be retrieved and viewed.

In some embodiments, caregivers may input the information relating to the patients into the EHR system 102. In some embodiments, information relating to the patients may be automatically transmitted to the EHR system 102, such as by a pulse oximeter, a blood pressure monitor, or any other type of health monitoring device communicatively coupled to the EHR system 102. Such health monitoring devices may be communicatively coupled to the EHR system 102 using a wired communication technology (including but not limited to Ethernet, serial cables, USB, or FireWire), a wireless communication technology (including but not limited to Bluetooth, Wi-Fi, WiMAX, 3G, 4G, 5G, or LTE), or combinations thereof.

A physician 108 may interact with the EHR system 102 using a prioritization computing device 106. The prioritization computing device 106 may be any type of computing device configured to be communicatively coupled to the EHR system 102 and to present information from the EHR system 102 to the physician 108. The illustrated embodiment of the prioritization computing device 106 is a tablet computing device, but in other embodiments, the prioritization computing device 106 may be a desktop computing device, a laptop computing device, a smartphone computing device, or any other type of computing device suitable for presenting a prioritization interface as described herein.

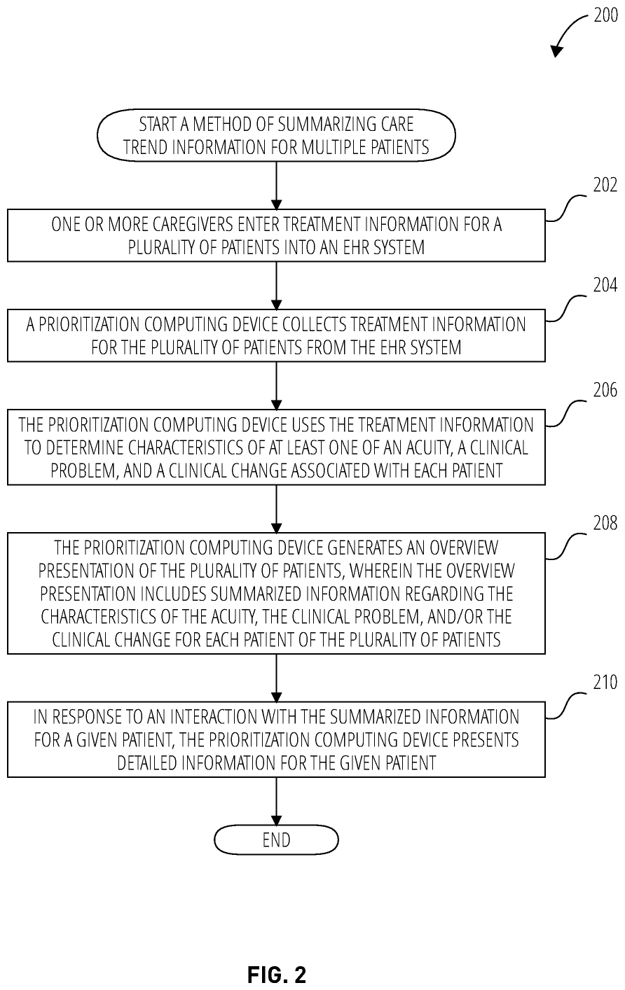

FIG. 2 is a flowchart that illustrates a non-limiting example embodiment of a method of summarizing care trend information for multiple patients according to various aspects of the present disclosure. As stated above, though the EHR system 102 is useful in storing and viewing information for individual patients, typical EHR systems 102 do not provide the ability to view summary information that allows physicians 108 to easily and quickly compare the conditions of multiple patients in order to make decisions regarding care prioritization. By using the method 200, the health care environment 100 is enhanced by providing such a view of summary information, and the physician 108 is enabled to make such decisions quickly and easily without having to struggle through individual patient records within the EHR system 102.

From a start block, the method 200 proceeds to block 202, where one or more caregivers enter treatment information for a plurality of patients into an EHR system 102. The treatment information may include lab test results for the patients, results of diagnostic evaluations for the patients, documentation of treatments received by the patients, documented communications regarding the patients, and/or other types of treatment information.

At block 204, a prioritization computing device 106 collects treatment information for the plurality of patients from the EHR system 102. In some embodiments, the prioritization computing device 106 may query the EHR system 102 via any suitable communication technology supported by the EHR system 102 to retrieve the treatment information for the plurality of patients. In some embodiments, the prioritization computing device 106 may collect treatment information for a predetermined period of time, such as a preceding 24-hour period. In some embodiments, other predetermined periods of time may be used.

At block 206, the prioritization computing device 106 uses the treatment information to determine characteristics of at least one of an acuity, a clinical problem, and a clinical change associated with each patient. In some embodiments, the prioritization computing device 106 may determine more than one of the acuity, the clinical problem, and the clinical change for each patient. In some embodiments, whether the prioritization computing device 106 determines characteristics of an acuity, a clinical problem, or a clinical change may depend on a type of interface chosen by the physician 108, as illustrated and described in further detail below. In some embodiments, the characteristics may include aspects including, but not limited to, one or more of a count of instances of treatment information (e.g., a number of diagnostic tests, a number of documented communications), presence or absence of a given clinical problem, a rate of change of a diagnostic test result, and an amount of deviation of a diagnostic test result from a normal result.

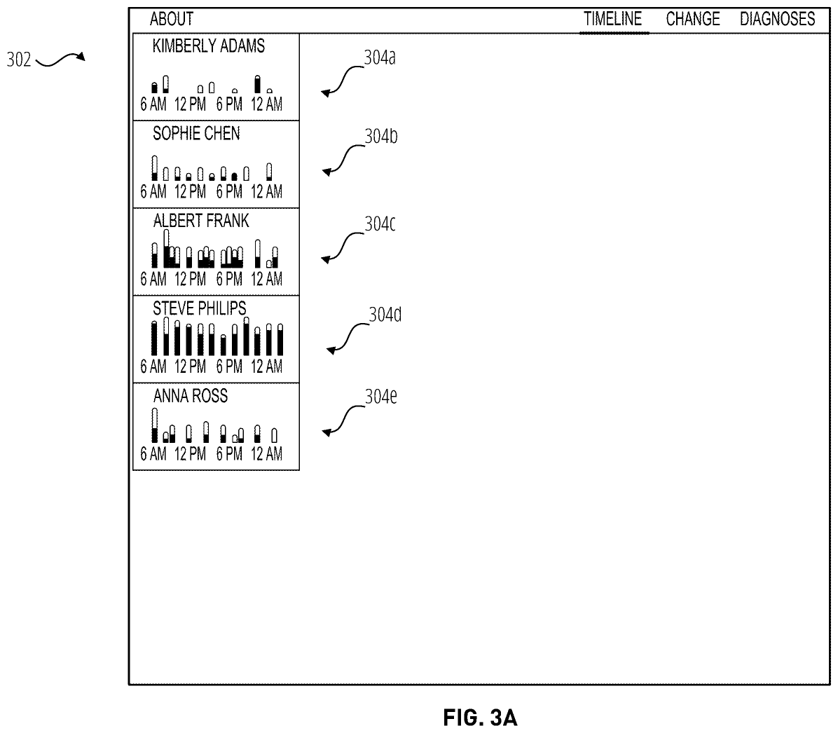

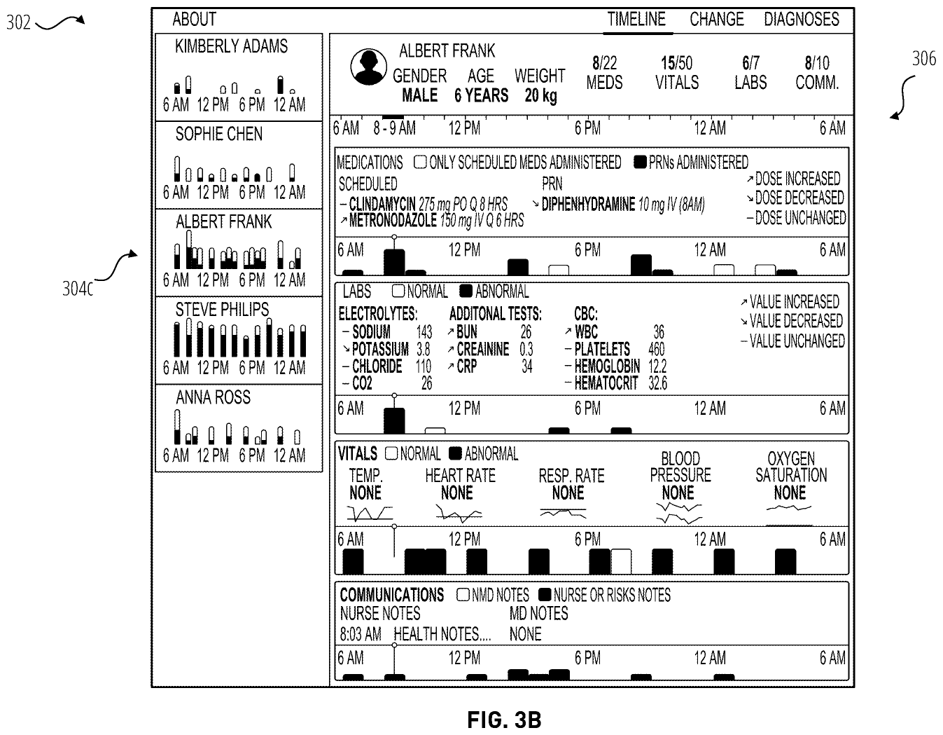

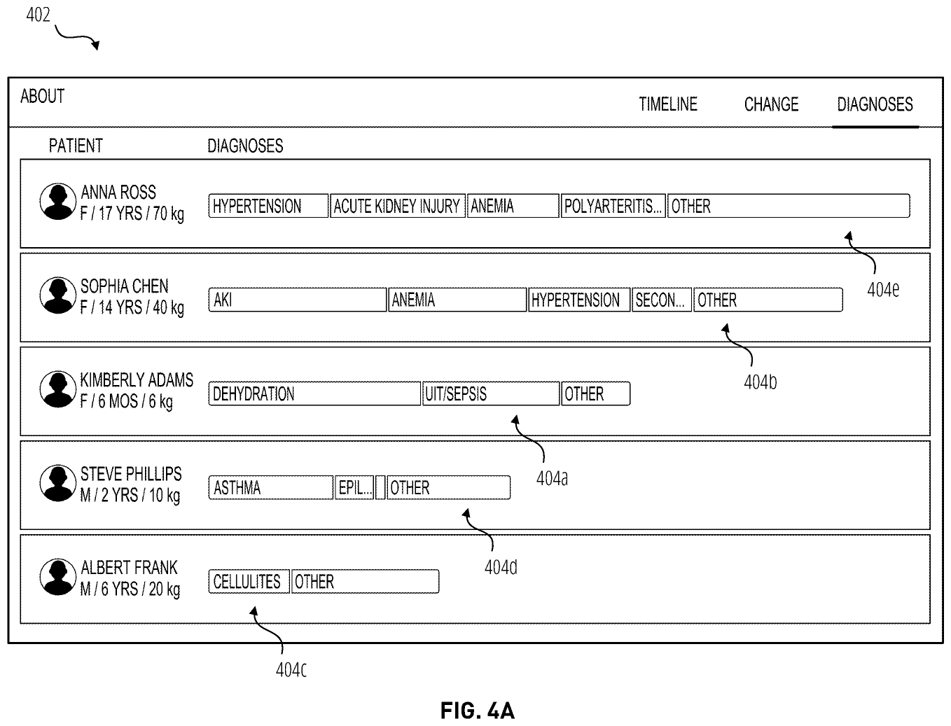

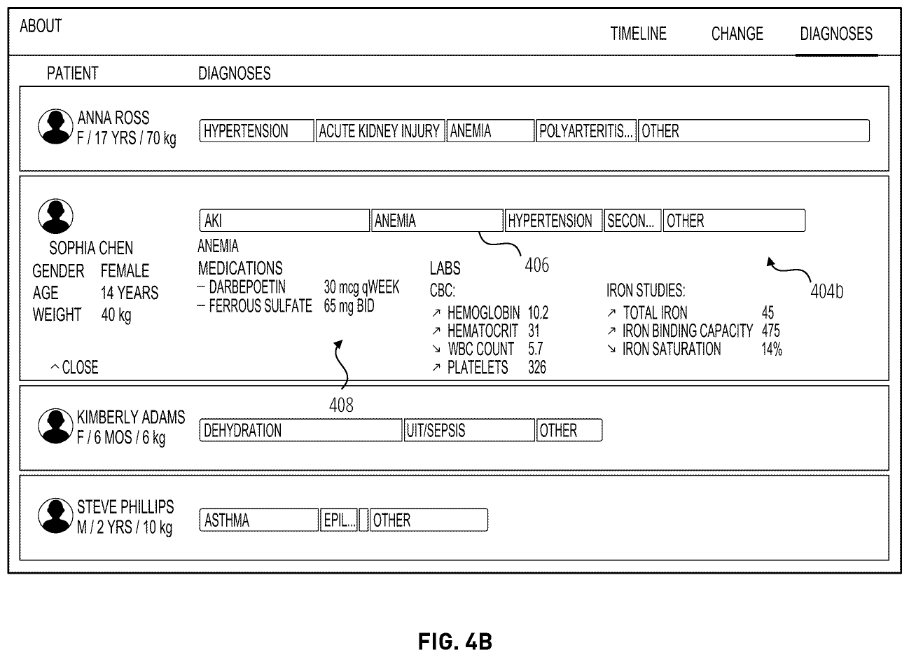

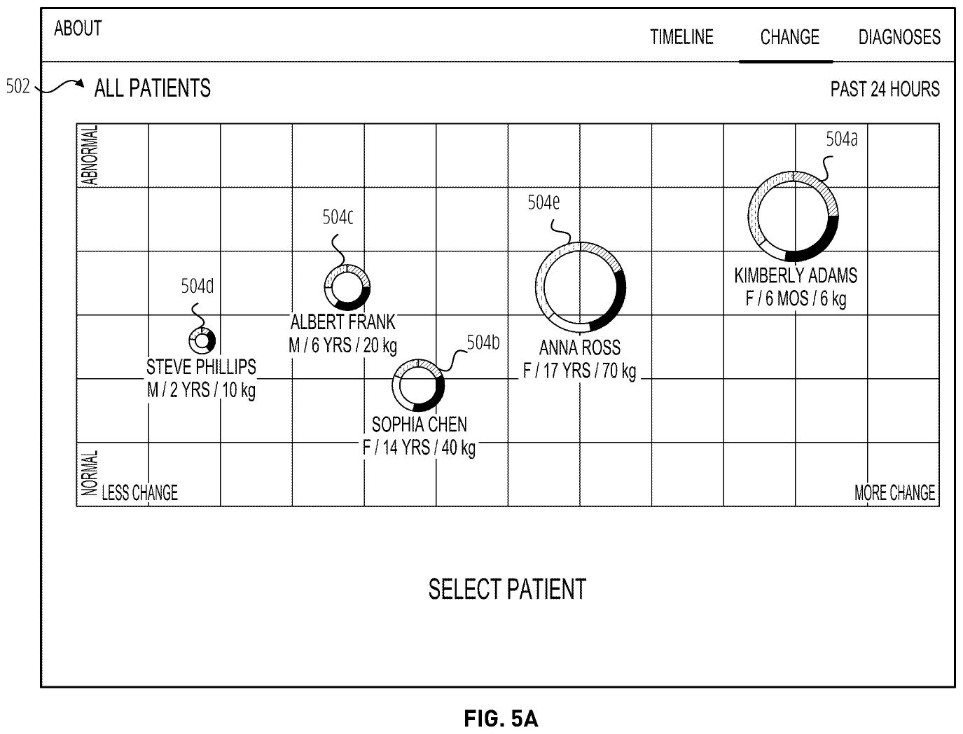

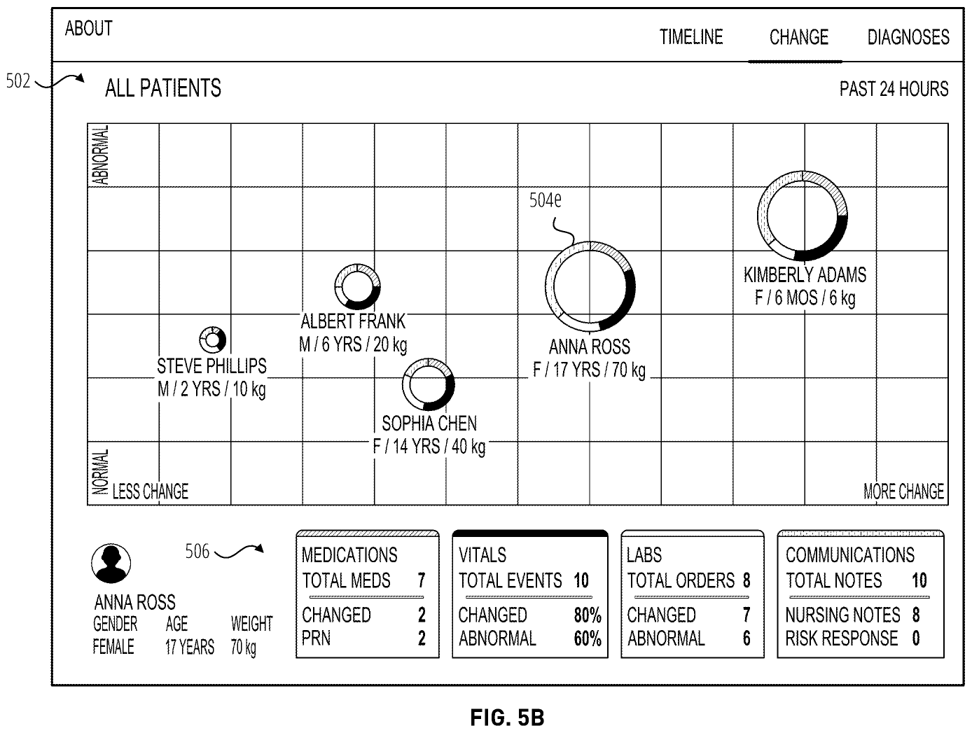

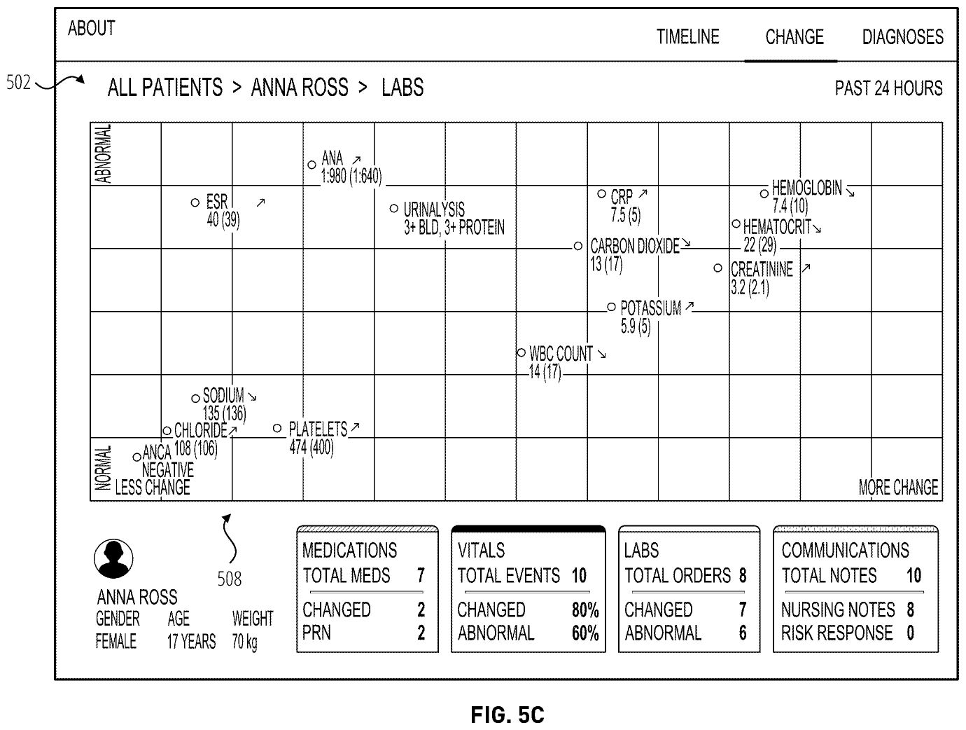

At block 208, the prioritization computing device 106 generates an overview presentation of the plurality of patients, wherein the overview presentation includes summarized information regarding the characteristics of the acuity, the clinical problem, and/or the clinical change for each patient of the plurality of patients. The overview presentation may include summarized information for a subset of patients tracked by the EHR system 102. In some embodiments, the prioritization computing device 106 may sort patients by severity, and the overview presentation may present the most severe patients, or may sort the patients by severity such that the physician 108 may scroll past the patients sorted as being most severe to reach summarized information for less severe patients. In some embodiments, the physician 108 may be able to interact with the prioritization computing device 106 to switch between different types of overview presentations, such as switching between an acuity interface (FIG. 3A-FIG. 3B), a clinical problem interface (FIG. 4A-FIG. 4B), and a clinical change interface (FIG. 5A-FIG. 5C) as illustrated and described in further detail below.

At block 210, in response to an interaction with the summarized information for a given patient, the prioritization computing device presents detailed information for the given patient. Various embodiments of the presentation of detailed information in an acuity interface, a clinical problem interface, and a clinical change interface are also illustrated and descried in further detail below. In some embodiments, block 210 may be skipped if no interaction from the physician 108 is detected by the prioritization computing device 106.

The method 200 then proceeds to an end block and terminates. One will note that in some embodiments, the method 200 may perform the actions of block 210 multiple times before advancing to the end block, and/or may loop back and present updated or refreshed overview presentations or detailed information before terminating.

In the description of method 200, certain actions are described as being performed by the prioritization computing device 106 or EHR system 102. In some embodiments, actions described as being performed by the prioritization computing device 106 may be performed instead by the EHR system 102, including but not limited to gathering information and generating interfaces, and the prioritization computing device 106 may be used to simply present the interfaces generated by the EHR system 102. In some embodiments, it may be desirable to have the information gathered from the EHR system 102 by the prioritization computing device 106, and to have the interfaces generated by prioritization computing device 106 as described above, because such embodiments would allow the prioritization computing device 106 to present overview presentations without requiring changes to existing EHR systems 102.

It has been determined that physicians 108 utilize certain mental models when prioritizing their patients: (1) acuity, (2) a patient's clinical problem list, and (3) clinical change. Accordingly, embodiments of the present disclosure present overview presentations of information from the EHR system 102 that is organized around at least one of these mental models. The non-limiting example presentations illustrated and described below leverage Schniederman's Visual Information-Seeking Mantra: “overview first, zoom and filter, then details on demand,” by first presenting the physician 108 an overview of all of the patients based around one of these mental models (as described at block 208). Each presentation illustrated below may then provide patient-level detailed information (as described at block 210) in four example categories: medications, laboratory tests, vital signs, and clinical notes/communications. In other embodiments, more, fewer, and/or different categories may be used for patient-level details.

FIG. 3A illustrates a non-limiting example embodiment of an overview presentation organized by acuity according to various aspects of the present disclosure.

Acuity represents the severity of an illness. Patients with higher levels of acuity tend to be sicker, have more abnormal test results, are less stable, and typically require more interventions, evaluations and treatments. While others have created a variety of tools to identify patients at risk of clinical deterioration, we sought to explore how to communicate the concept of acuity by highlighting the level of patient activity. In this regard, activity represents the number of diagnostic evaluations performed on an individual (e.g. lab tests, radiology examinations, vital sign measurements, etc.), treatments they receive, and the frequency of verbal and documented communications between various clinical care team members. A higher acuity patient (i.e. sicker, or more in need of care) will have higher levels of activity compared to a lower acuity patient. In addition, it is helpful for the physician 108 to know how much of this activity is abnormal, as higher acuity patients also tend to have a higher proportion of abnormal activity compared to those with lower acuity.

The acuity interface 302 illustrated in FIG. 3A organizes information about each patient through an acuity lens, displaying the amount and pattern of activity (both normal and abnormal) over the course of a 24-hour period. A first bar chart 304a is associated with patient 104a, a second bar chart 304b is associated with patient 104b, a third bar chart 304c is associated with patient 104c, a fourth bar chart 304d is associated with patient 104d, and a fifth bar chart 304e is associated with patient 104e.

Each bar chart includes vertical bars, wherein a horizontal position of each bar represents a time period during which the associated activities were conducted, and a height of each bar represents a number of activities during the time period. In some embodiments, the activities may include diagnostic evaluations performed on the patient, treatments received by the patient, documented communications regarding the patient, or other types of activities. In some embodiments, solid portions of the bars may be associated with numbers of abnormal results, while hollow portions of the bars may be associated with numbers of normal results.

By reviewing the height and frequency of the bars, a physician 108 can quickly determine differences in activity across all five patients. As a rough approximation, a physician 108 may determine that a patient whose bar chart exhibits a greater “area under the curve”, or that includes more, higher bars, should be prioritized over a patient whose bar chart includes fewer or shorter bars.

FIG. 3B illustrates a non-limiting example embodiment of detailed information presented after an interaction with a bar chart illustrated in FIG. 3A. In FIG. 3B, the physician 108 had interacted with the bar chart 304c for patient 104c. This caused detailed information 306 to be presented at the right side of the interface, showing the detailed information retrieved from the EHR system 102 for the patient 104c across four different clinical categories identified as important: medications, laboratory tests, vital signs, and clinical notes/communications. Selecting a specific time point on the display may cause the acuity interface 302 to present an indication of what activity took place at that time in each of the clinical categories.

FIG. 4A illustrates a non-limiting example embodiment of an overview presentation organized by clinical problem, according to various aspects of the present disclosure.

Patients typically get admitted to a care facility due to a clinical deterioration or problem. Caregivers gather information to identify patterns that help to classify patients as having a specific illness, diagnosis, or problem. With the problem identified, physicians 108 can provide appropriate therapies to treat the patient. This is a part of patient management, as a patient's problem or problems will dictate their prognosis, response to treatment, and expected course over time. Physicians 108 process details about their patients in the context of their clinical problems and use the clinical problems as a basis to communicate with other physicians. To leverage these facts, the clinical problem interface 402 illustrated in FIG. 4A organizes data by a patient's list of clinical problems to support clinical decision making.

The clinical problem interface 402 displays a list of problems for each of the patients, with each problem being represented by a horizontal line. As shown, a horizontal line 404a is provided for patient 104a, a horizontal line 404b is provided for patient 104b, a horizontal line 404c is provided for patient 104c, a horizontal line 404d is provided for patient 104d, and a horizontal line 404e is provided for patient 104e. One will note that each horizontal line is divided into one or more segments. Each segment of each horizontal line represents a clinical problem for the patient.

In some embodiments, the length of each segment, and therefore the total length of each horizontal line, is based on activity retrieved from the EHR system 102 relating to a clinical problem represented by each segment within a predetermined time period (such as the preceding 24 hours). For example, in some embodiments, the length of each segment may be determined based on one or more of a number of treatments the patient received for the clinical problem, a number of tests the patient received relating to the clinical problem, a determination of whether one or more of the tests resulted in abnormal results (and/or the amount of deviation from a normal range), and how the results of one or more tests have changed over a predetermined time period (such as 24 hours).

By comparing lengths of the horizontal lines, a physician 108 can prioritize care for the patients. In some embodiments, the physician 108 may instead notice a particularly long segment that relates to a particularly serious clinical problem, and may prioritize care for a patient based on that particularly long segment. One will note that the horizontal lines have been sorted from a longest horizontal line to a shortest horizontal line to aid in prioritization.

FIG. 4B illustrates a non-limiting example embodiment of detailed information presented after an interaction with a segment associated with a specific clinical problem for a given patient in the clinical problem interface 402 illustrated in FIG. 4A. In FIG. 4B, the physician 108 had interacted with the anemia segment 406 of the horizontal line 404b associated with patient 104b. This caused detailed information 408 to be presented relating to the associated clinical problem below the horizontal line 404b, showing the detailed information retrieved from the EHR system 102 for the clinical problem for the patient 104b.

FIG. 5A illustrates a non-limiting example embodiment of an overview presentation organized by clinical change, according to various aspects of the present disclosure.

Clinical change is the third clinical prioritization concept used by physicians. Following various disease specific parameters allows physicians to determine how a patient's disease progresses over time and, more importantly, how a patient's disease responds to various therapies and treatments. Change can result in a clinical improvement or a patient deteriorating, suggesting the importance of communicating directionality along with change. One way to accomplish this is by providing details on how abnormal a patient's results are. Therefore, by combining these two concepts, there is potential to quickly describe the status of an individual patient and give a sense of how quickly one needs to intervene in their care.

The clinical change interface 502 leverages the three concepts of change, abnormality, and activity to describe the progression of patients over a previous predetermined time period, such as a 24-hour time period. Each patient is represented by a clinical change indicator, such as the clinical change circles illustrated in FIG. 5A and FIG. 5B. As shown, the patient 104a is represented by a clinical change circle 504a, the patient 104b is represented by a clinical change circle 504b, a patient 104c is represented by a clinical change circle 504c, a patient 104d is represented by a clinical change circle 504d, and a patient 104e is represented by a clinical change circle 504e.

The size and position of each clinical change circle can be used separately to provide information to the physician 108. Regarding size, the diameter of each clinical change circle is directly related to an amount of activity that took place for the associated patient over the previous predetermined time period. In addition, each circle is divided into four segments, each segment representing one of four clinical categories: medications, laboratory results, vital signs, and communications. The size of each segment represents how the associated clinical category contributes to the overall activity of the patient during the predetermined time period.

Regarding position, the clinical change interface 502 provides a cartesian coordinate system. An amount of clinical change is illustrated on a horizontal axis, and an amount of abnormality is illustrated on a vertical axis. Accordingly, the farther to the left a clinical change circle appears, the less change has taken place, and the farther to the right a clinical change circle appears, the more change has taken place. Likewise, the higher a clinical change circle appears, the more abnormal the test results had been, while the lower a clinical change circle appears, the less abnormal the test results had been. This leads to being able to describe the lower left quadrant as “stable normal,” the upper left quadrant as “stable abnormal,” the lower right quadrant as “unstable normal,” and the upper right quadrant as “unstable abnormal,” with the location and size of a clinical change circle roughly indicating the status of the associated patient in these quadrants.

FIG. 5B illustrates a non-limiting example embodiment of summarized information for a given patient presented after interaction with a clinical change circle for a given patient in the clinical change interface 502 illustrated in FIG. 5A. In FIG. 5B, the physician 108 had interacted with the clinical change circle 504e for patient 104e. This caused summarized information 506 to be presented, wherein a numerical version of each of the categories represented in the clinical change circle 504e is provided. One will note that the shading at the top of each category in the display of summarized information 506 matches the shading of the associated segments in the clinical change circle 504e in order to clearly indicate the link between the summarized information 506 and the clinical change circle 504e.

Though FIG. 5A and FIG. 5B illustrate summarized information using clinical change circles as clinical change indicators, in some embodiments, shapes other than circles may be used as clinical change indicators. For example, in some embodiments, a square, a box, or another shape may be used as a clinical change indicator in a similar way as described above with respect to the clinical change circles. In some embodiments, the size of the clinical change indicator may not encode any information, and the relative severity of each patient may be indicated by the location of the clinical change indicator within the cartesian coordinate system alone.

FIG. 5C illustrates a non-limiting example embodiment of detailed information for a given patient presented after interaction with the summarized information for a given patient in the clinical change interface 502 illustrated in FIG. 5B. As shown, after interacting with the summarized information for the “Labs” category as illustrated in FIG. 5B, the clinical change interface 502 presents detailed information 508 for the “Labs” category using the same cartesian coordinate system from FIG. 5A and FIG. 5B. The detailed information 508 also indicates trend information for each element of presented detailed information, so that by reviewing the detailed information 508, the physician 108 can quickly determine for each of the lab results (1) whether the result is normal or abnormal based on its vertical position, (2) how much the result has changed over the predetermined time period based on its horizontal position, and (3) whether the result is trending up or down based on the arrow located near each displayed test result.

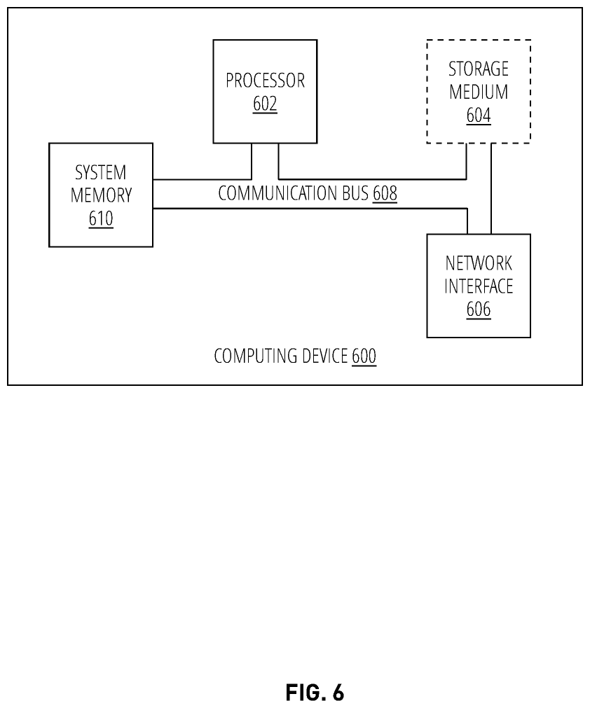

FIG. 6 is a block diagram that illustrates aspects of an exemplary computing device 600 appropriate for use as a computing device of the present disclosure. While multiple different types of computing devices were discussed above, the exemplary computing device 600 describes various elements that are common to many different types of computing devices. While FIG. 6 is described with reference to a computing device that is implemented as a device on a network, the description below is applicable to servers, personal computers, mobile phones, smart phones, tablet computers, embedded computing devices, and other devices that may be used to implement portions of embodiments of the present disclosure. Some embodiments of a computing device may be implemented in or may include an application-specific integrated circuit (ASIC), a field-programmable gate array (FPGA), or other customized device. Moreover, those of ordinary skill in the art and others will recognize that the computing device 600 may be any one of any number of currently available or yet to be developed devices.

In its most basic configuration, the computing device 600 includes at least one processor 602 and a system memory 610 connected by a communication bus 608. Depending on the exact configuration and type of device, the system memory 610 may be volatile or nonvolatile memory, such as read only memory (“ROM”), random access memory (“RAM”), EEPROM, flash memory, or similar memory technology. Those of ordinary skill in the art and others will recognize that system memory 610 typically stores data and/or program modules that are immediately accessible to and/or currently being operated on by the processor 602. In this regard, the processor 602 may serve as a computational center of the computing device 600 by supporting the execution of instructions.

As further illustrated in FIG. 6, the computing device 600 may include a network interface 606 comprising one or more components for communicating with other devices over a network. Embodiments of the present disclosure may access basic services that utilize the network interface 606 to perform communications using common network protocols. The network interface 606 may also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface 606 illustrated in FIG. 6 may represent one or more wireless interfaces or physical communication interfaces described and illustrated above with respect to particular components of the computing device 600.

In the exemplary embodiment depicted in FIG. 6, the computing device 600 also includes a storage medium 604. However, services may be accessed using a computing device that does not include means for persisting data to a local storage medium. Therefore, the storage medium 604 depicted in FIG. 6 is represented with a dashed line to indicate that the storage medium 604 is optional. In any event, the storage medium 604 may be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computing devices that include a processor 602, system memory 610, communication bus 608, storage medium 604, and network interface 606 are known and commercially available. For ease of illustration and because it is not important for an understanding of the claimed subject matter, FIG. 6 does not show some of the typical components of many computing devices. In this regard, the computing device 600 may include input devices, such as a keyboard, keypad, mouse, microphone, touch input device, touch screen, tablet, and/or the like. Such input devices may be coupled to the computing device 600 by wired or wireless connections including RF, infrared, serial, parallel, Bluetooth, Bluetooth low energy, USB, or other suitable connections protocols using wireless or physical connections. Similarly, the computing device 600 may also include output devices such as a display, speakers, printer, etc. Since these devices are well known in the art, they are not illustrated or described further herein.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of generating a user interface for summarizing care trend information for multiple patients, the method comprising:
for each patient of a plurality of patients, collecting treatment information for the patient from an electronic health record system, wherein the treatment information reflects at least one of a patient acuity, a patient clinical problem, and a clinical change in the patient;
generating an overview presentation of the plurality of patients, wherein the overview presentation includes summarized information for each patient of the plurality of patients; and
displaying the overview presentation to a user.

2. The method of claim 1, wherein the treatment information includes information regarding results of at least one diagnostic test.

3. The method of claim 2, wherein the information regarding results of at least one diagnostic test includes information regarding multiple administrations of the at least one diagnostic test over time.

4. The method of claim 1, wherein the overview presentation is organized by acuity.

5. The method of claim 4, wherein the overview presentation organized by acuity includes a bar chart;
wherein a height of each bar represents at least one of a number of diagnostic evaluations performed on the patient, a number of treatments received by the patient, and a frequency of documented communications regarding the patient; and
wherein the height of each bar and a frequency of the bars provides a visual approximation of acuity.

6. The method of claim 1, wherein the overview presentation is organized by clinical problem.

7. The method of claim 6, wherein the overview presentation organized by clinical problem includes a horizontal line for each patient;
wherein the horizontal line comprises one or more segments; and
wherein each segment of the one or more segments represents a clinical problem for the patient.

8. The method of claim 7, wherein a length of each segment is based on one or more of a number of treatments the patient received for the clinical problem, a number of tests the patient received for the clinical problem, a determination of whether the tests were abnormal, and how the tests have changed over a predetermined time period.

9. The method of claim 1, wherein the overview presentation is organized by clinical change.

10. The method of claim 9, wherein the overview presentation organized by clinical change includes a coordinate system with clinical change represented on a horizontal axis and abnormality represented on a vertical axis; and
wherein the overview presentation organized by clinical change includes a clinical change indicator for each patient placed in appropriate locations in the coordinate system.

11. The method of claim 10, wherein a size of each clinical change indicator is based on an amount of activity that took place for the patient over a predetermined time period.

12. The method of claim 10, wherein each clinical change indicator is a circle divided into a first segment that represents medications, a second segment that represents laboratory results, a third segment that represents vital signs, and a fourth segment that represents communications.

13. The method of claim 1, further comprising:
detecting a user interaction with the summarized information for a given patient; and presenting detailed information for the given patient in response to the user interaction.

14. A non-transitory computer-readable medium having computer-executable instructions stored thereon that, in response to execution by one or more processors of a computing device, cause the computing device to perform actions for generating a user interface for summarizing care trend information for multiple patients, the actions comprising:
for each patient of a plurality of patients, collecting treatment information for the patient from an electronic medical record system, wherein the treatment information reflects at least one of a patient acuity, a patient clinical problem, and a clinical change in the patient;
generating an overview presentation of the plurality of patients, wherein the overview presentation includes summarized information for each patient of the plurality of patients; and
displaying the overview presentation to a user.

15. The computer-readable medium of claim 14, wherein the treatment information includes information regarding results of at least one diagnostic test, and wherein the information regarding the results of the at least one diagnostic test includes information regarding multiple administrations of the at least one diagnostic test over time.

16. The computer-readable medium of claim 14, wherein the overview presentation is organized by acuity;
wherein the overview presentation organized by acuity includes a bar chart;
wherein a height of each bar represents at least one of a number of diagnostic evaluations performed on the patient, a number of treatments received by the patient, and a frequency of documented communications regarding the patient; and
wherein the height of each bar and a frequency of the bars provides a visual approximation of acuity.

17. The computer-readable medium of claim 14, wherein the overview presentation is organized by clinical problem;
wherein the overview presentation organized by clinical problem includes a horizontal line for each patient;
wherein the horizontal line comprises one or more segments; and
wherein each segment of the one or more segments represents a clinical problem for the patient.

18. The computer-readable medium of claim 17, wherein a length of each segment is based on one or more of a number of treatments the patient received for the clinical problem, a number of tests the patient received for the clinical problem, a determination of whether the tests were abnormal, and how the tests have changed over a predetermined time period.

19. The computer-readable medium of claim 14, wherein the overview presentation is organized by clinical change;
wherein the overview presentation organized by clinical change includes a coordinate system with clinical change represented on a horizontal axis and abnormality represented on a vertical axis;
wherein the overview presentation organized by clinical change includes a clinical change indicator for each patient placed in appropriate locations in the coordinate system; and
wherein a size of each clinical change indicator is based on an amount of activity that took place for the patient over a predetermined time period.

20. The computer-readable medium of claim 19, wherein each clinical change indicator is a circle divided into a first segment that represents medications, a second segment that represents laboratory results, a third segment that represents vital signs, and a fourth segment that represents communications.
